(19) United States
(12) Patent Application Publication
(10) Pub. No.: US 2020/0056796 A1
(43) Pub. Date: Feb. 20, 2020

(54) CARBON MONOXIDE DETECTION AND EXPULSION SYSTEM

(71) Applicant: Timothy D. Grimes, Dracut, MA (US)

(72) Inventor: Timothy D. Grimes, Dracut, MA (US)

(21) Appl. No.: 16/536,807

(22) Filed: Aug. 9, 2019

Related U.S. Application Data
(60) Provisional application No. 62/718,686, filed on Aug. 14, 2018.

(51) Int. Cl. F24F 11/00; G01N 33/00; G08B 21/14; G08B 3/10

(52) U.S. Cl. CPC F24F 11/0001; G01N 33/004; F24F 2110/72; G08B 3/10; G08B 21/14

(57) ABSTRACT

Disclosed is a system and method for carbon monoxide detection and expulsion thereof. Embodiments of the invention provide a carbon monoxide detection and expulsion system comprising a carbon monoxide detector that is configured to detect a level of carbon monoxide gas and output an activation signal when a detected level of carbon monoxide gas exceeds a predetermined threshold level, and an exhaust fan coupled to the carbon monoxide detector, the exhaust fan configured to turn on upon receiving the activation signal and to expel the carbon monoxide gas.

### CROSS-REFERENCE TO RELATED APPLICATIONS

This application claims the benefit under 35 U.S.C. § 119(e) of co-pending U.S. Provisional Application No. 62/718,686 filed on Aug. 14, 2018 and titled “CARBON MONOXIDE DETECTION AND EXPULSION SYSTEM,” which is incorporated by reference herein in its entirety for all purposes.

### FIELD

Aspects disclosed herein relate to carbon monoxide detection within enclosed units and expulsion thereof.

### BACKGROUND

In addition to other sources, carbon monoxide is produced from heating appliances and vehicles that are dependent on fossil fuels to function. The specific gravity of carbon monoxide is slightly lighter than that of air, and carbon monoxide will accumulate at ceiling level and will not dissipate unless there is air movement. The accumulation of carbon monoxide within enclosed units is hazardous as it could lead to carbon monoxide poisoning.

### SUMMARY

The system described herein reduces the hazards of carbon monoxide by detecting a level of carbon monoxide within an enclosed unit and activating an exhaust fan system within the enclosed unit to expel carbon monoxide to an exterior of the enclosed unit until a level of carbon monoxide within the enclosed unit drops below a predetermined threshold level.

An aspect of the present invention is directed a carbon monoxide detection and expulsion system comprising a carbon monoxide detector that is configured to detect a level of carbon monoxide gas and output an activation signal when a detected level of carbon monoxide gas exceeds a predetermined threshold level, and an exhaust fan coupled to the carbon monoxide detector, the exhaust fan configured to turn on upon receiving the activation signal and to expel the carbon monoxide gas.

According to one embodiment, the carbon monoxide detection and expulsion system is configured to be installed within an enclosed structure, and wherein the exhaust fan is configured to expel the carbon monoxide gas to an exterior of the enclosed structure. According to another embodiment, the exhaust fan is connected to a ductwork of the enclosed structure, wherein the ductwork connects an interior of the enclosed structure to the exterior of the enclosed structure and provides a passage for the carbon monoxide gas to be expelled from the interior of the enclosed structure to the exterior of the enclosed structure when the exhaust fan is activated. According to one embodiment, the ductwork is any one or a combination of a Polyvinyl Chloride (PVC) pipe, aluminum flex tube, and a solid aluminum pipe.

According to another embodiment, the carbon monoxide detector is configured to output a deactivation signal when the detected level of carbon monoxide gas falls below the predetermined threshold level.

According to one embodiment, the carbon monoxide detector is configured to measure the level of carbon monoxide gas in parts per million (PPM) and compare the measured level of carbon monoxide gas to a predetermined PPM threshold level. According to another embodiment, the carbon monoxide detector is configured to output a signal to the exhaust fan to be tuned on or off based on whether the measured level of carbon monoxide gas exceeds the predetermined PPM threshold level.

According to another embodiment, the exhaust fan is configured to have a cubic feet per minute (CFM) flow capacity that is determined based on a square footage of an area being protected. According to an embodiment, the exhaust fan is configured to be ceiling-mounted or wall-mounted. According to another embodiment, the exhaust fan is any or all of moisture-resistant, steam-resistant, and mold-resistant.

According to one embodiment, the carbon monoxide detection and expulsion system further comprises a sound alarm connected to the carbon monoxide detector, the carbon monoxide detector configured to send an activation signal to the sound alarm when the detected level of carbon monoxide gas exceeds the predetermined threshold level.

According to another embodiment, the carbon monoxide detection and expulsion system further comprises a power supply relay module connected to the carbon monoxide detector and the exhaust fan. The carbon monoxide detector upon detecting a certain threshold level of carbon monoxide sends an activation signal to the power supply relay module. The power supply relay module is configured to receive the activation signal and send a fan activation signal to the exhaust fan to turn on the exhaust fan.

Another aspect of the present invention is directed to a method of detecting and expelling carbon monoxide, the method comprising detecting a level of carbon monoxide, comparing the detected level of carbon monoxide to a threshold level, turning on an exhaust fan when the detected level of carbon monoxide exceeds the threshold level, and expelling the carbon monoxide when the detected level of carbon monoxide exceeds the threshold level.

According to one embodiment, detecting the level of carbon monoxide includes detecting the level of carbon monoxide within an enclosed volume. According to another embodiment, comparing the detected level of carbon monoxide to the threshold level includes comparing the detected level of carbon monoxide within the enclosed volume to the threshold level.

According to one embodiment, turning on the exhaust fan includes sending an activation signal to the exhaust fan when the detected level of carbon monoxide exceeds the threshold level.

According to another embodiment, the method further comprises activating a sound alarm when the detected level of carbon monoxide exceeds the threshold level.

According to one embodiment, detecting the level of carbon monoxide includes measuring the level of carbon monoxide in parts per million.

According to another embodiment, the method further comprises turning off the exhaust fan when the detected level of carbon monoxide falls below the threshold level. According to on embodiment, turning off the exhaust fan includes sending a deactivation signal to the exhaust fan when the detected level of carbon monoxide falls below the threshold level.

### BRIEF DESCRIPTION OF THE DRAWINGS

FIG. 1A is a schematic diagram of a carbon monoxide detection and expulsion system according to one embodiment;

FIG. 1B is a schematic diagram of a carbon monoxide detection and expulsion system according to another embodiment;

FIG. 2 is a schematic diagram of a carbon monoxide detection and expulsion system for residential units according to one embodiment; and

FIG. 3 is a schematic diagram of a carbon monoxide detection and expulsion system for residential units according to another embodiment.

### DETAILED DESCRIPTION

Various aspects of the carbon monoxide detection and expulsion system will now be discussed in detail with reference to the accompanying drawings. It is to be appreciated that this system is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having”, “containing,” “involving,” and variations thereof herein, is meant to encompass the items listed thereafter and equivalents thereof as well as additional items.

At least some embodiments herein provide a carbon monoxide detection and expulsion system that can help eliminate carbon monoxide poisoning within residential homes, buildings, or structures where carbon monoxide gas can build up, for example structures where heating equipment are in use and/or that have enclosed areas for vehicle storage.

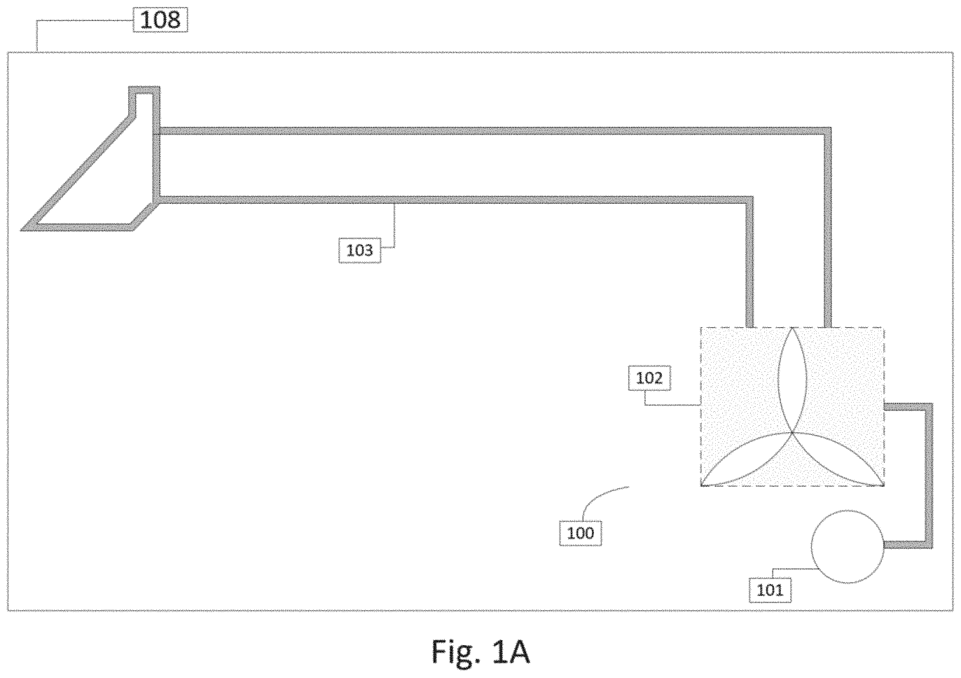

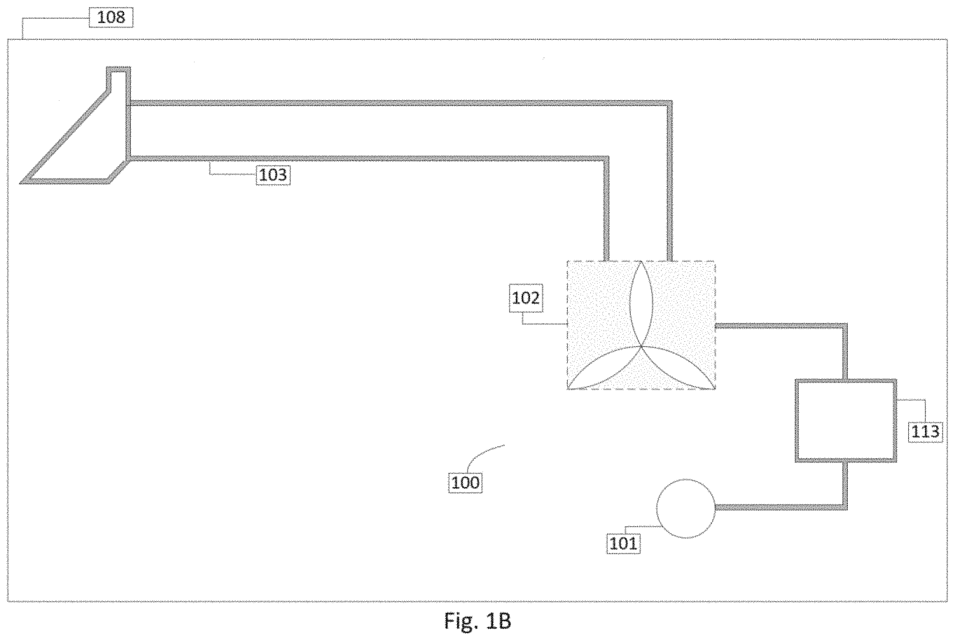

FIG. 1 is a schematic diagram of one embodiment of a carbon monoxide detection and expulsion system 100. The carbon monoxide detection and expulsion system 100 is comprised of a carbon monoxide detector 101 and an exhaust fan 102. According to one embodiment, the exhaust fan 102 can be coupled to ductwork 103, such as for example a polyvinyl chloride (PVC) pipe, an aluminum flex hose, a solid aluminum pipe, sheet metal ductwork, or any other structure known to those of skill in the art. In operation, the carbon monoxide detector 101 measures a level of carbon monoxide. According to an embodiment, the carbon monoxide detector can be configured to provide a determination of the level of carbon monoxide in parts per million (PPM). When the measured level of carbon monoxide exceeds a specified threshold, the carbon monoxide detector 101 will send an activation signal. The activation signal is used by the system to turn the exhaust fan 102 on. The system is configured so that the exhaust fan 102 will run until a safe level of carbon monoxide is detected by the carbon monoxide detector 101, for example a measured PPM level of carbon monoxide that is below a specified PPM threshold level. For example, once the carbon monoxide drops below a certain threshold level, the carbon monoxide detector stops sending out the activation signal that is used to turn on the exhaust fan. In some embodiments, the carbon monoxide detector is set to activate at a threshold level of approximately 50 PPM, but it is to be appreciated that any threshold level can be selected. According to embodiments, the exhaust fan 102 is configured with cubic feet per minute (CFM) flow capacity that is determined based on the square footage of an area 108 being protected. It is to be appreciated that the exhaust fan 102 can be ceiling- or wall-mounted and can be coupled to existing ductwork. Alternatively, new piping or ductwork can be installed to which the exhaust fan 102 can be coupled. The exhaust fan 102 can also be any or all of moisture-resistant, steam-resistant, and mold-resistant. Once activated, the exhaust fan 102 will expel carbon monoxide to the exterior of a residential unit, a building, or a structure through the ductwork 103. The size of the ductwork 103 is determined by the CFM of the exhaust fan 102. According to one embodiment, either or both of the carbon monoxide detector 101 and the exhaust fan 102 are rated for a voltage of 120 Volts and have a battery backup.

FIG. 1B is a schematic diagram of an embodiment of the carbon monoxide detection and expulsion system 100 in which the carbon monoxide detector 101 is connected to the exhaust fan 102 using a relay power supply module 113. When the carbon monoxide detector 101 detects a level of carbon monoxide that exceeds a specified threshold level, the carbon monoxide detector 101 sends a signal, for example a voltage level above a specified threshold, to the relay power supply module 113. The relay power supply module 113 then sends an activation signal to the exhaust fan 102.

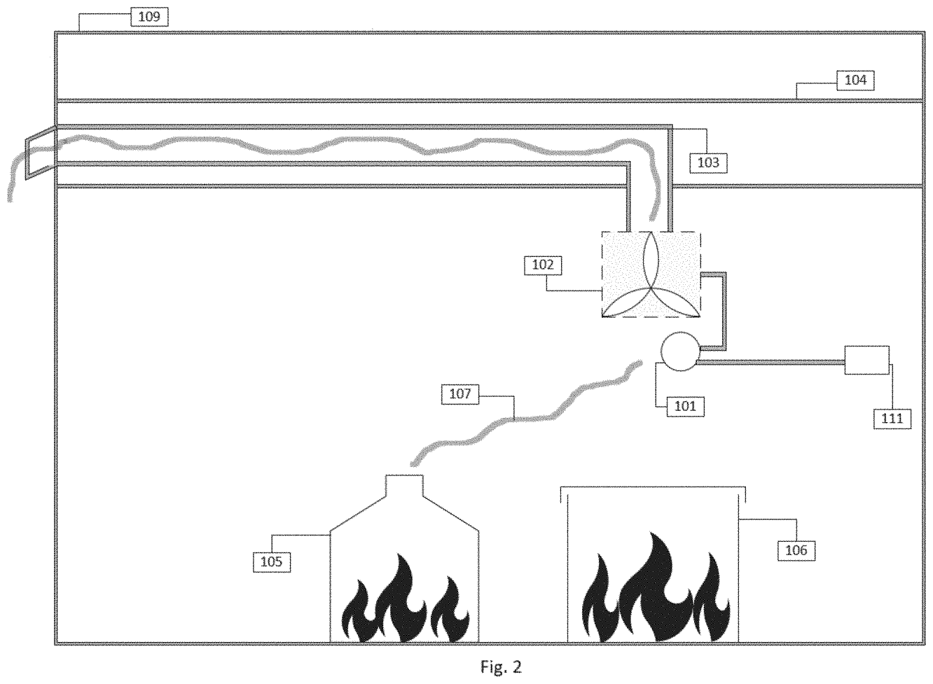

FIG. 2 is a system diagram that shows an application of the carbon monoxide detection and expulsion system 100 in a mechanical room 109. Mechanical rooms in residential units, such as the mechanical room 109, typically contain heating appliances fueled by fossil fuels. These heating appliances can malfunction, thereby producing carbon monoxide as a result. The mechanical room 109 of FIG. 2 is exemplary illustrated with both a hot water tank 105 and a furnace 106. Similar to the heating furnace 106, the hot water tank 105 can malfunction and produce a carbon monoxide exhaust 107. With this arrangement, the carbon monoxide detector 101 is configured to detect the level of carbon monoxide in the carbon monoxide exhaust 107 and to send a signal to activate the exhaust fan 102 when the detected level of carbon monoxide has exceeded a specified threshold level. Additionally, the carbon monoxide detector 101 can activate a sound alarm 111 when the detected level of carbon monoxide has exceeded the specified threshold level. Upon receiving an activation signal, the exhaust fan 102 turns on and expels the carbon monoxide exhaust 107 to an exterior of the mechanical room 109 through the ductwork 103, which by way of example can be installed in ceiling 104 of the mechanical room 109, until a safe level of carbon monoxide is reached. Once a safe level of carbon monoxide is reached, the system is configured to turn off the exhaust fan. However various modifications are anticipated also, such as continuing to keep the exhaust fan active even when the carbon monoxide level is below the predetermined threshold, until the fan is turned off manually (so as to require manual clearance).

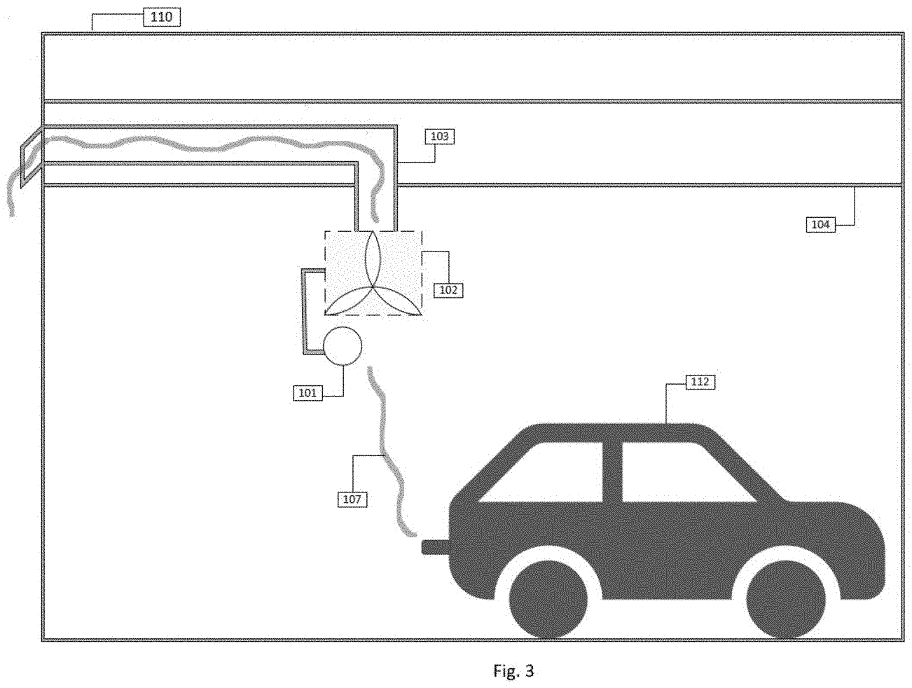

FIG. 3 is a schematic diagram that shows an application of the carbon monoxide detection and expulsion system 100 in an enclosed vehicle storage area 110. Vehicles in enclosed vehicle storage areas can emit high, potentially lethal, levels of carbon monoxide when running. When a vehicle 112 is running while in the enclosed vehicle storage area 110 and emits a carbon monoxide exhaust 107, the carbon monoxide detector 101 detects when the exhaust 107 exceeds a predetermined level or detects an amount of carbon monoxide present in PPM. When the carbon monoxide level in the carbon monoxide exhaust 107 exceeds a certain threshold level, the carbon monoxide detector sends an activation signal to the exhaust fan 102 to be turned on. The exhaust fan 102, once activated, expels the carbon monoxide 107 to an exterior of the enclosed vehicle storage area 110 through a ductwork 103, which can be in a ceiling 104 or a wall of the enclosed vehicle storage area 110, until a safe level of carbon monoxide is reached in the enclosed vehicle storage area 110. When a safe level of carbon monoxide level is achieved, for example, a level of carbon monoxide that is below the predetermined threshold level, the carbon monoxide detector 101 can send a deactivation signal to the exhaust fan 102. However various modifications are anticipated also, such as continuing to keep the exhaust fan active even when the carbon monoxide level is below the predetermined threshold level, until the fan is turned off manually (so as to require manual clearance).

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A carbon monoxide detection and expulsion system comprising:
a carbon monoxide detector that is configured to detect a level of carbon monoxide gas and output an activation signal when a detected level of carbon monoxide gas exceeds a predetermined threshold level; and
an exhaust fan coupled to the carbon monoxide detector, the exhaust fan configured to turn on upon receiving the activation signal and to expel the carbon monoxide gas.

2. The carbon monoxide detection and expulsion system of claim 1, wherein the carbon monoxide detection and expulsion system is configured to be installed within an enclosed structure, and wherein the exhaust fan is configured to expel the carbon monoxide gas to an exterior of the enclosed structure.

3. The carbon monoxide detection and expulsion system of claim 2, wherein the exhaust fan is connected to a ductwork of the enclosed structure, wherein the ductwork connects an interior of the enclosed structure to the exterior of the enclosed structure and provides a passage for the carbon monoxide gas to be expelled from the interior of the enclosed structure to the exterior of the enclosed structure when the exhaust fan is activated.

4. The carbon monoxide detection and expulsion system of claim 3, wherein the ductwork is any one of or a combination of a Polyvinyl Chloride (PVC) pipe, aluminum flex tube, and a solid aluminum pipe.

5. The carbon monoxide detection and expulsion system of claim 1, wherein the carbon monoxide detector is configured to output a deactivation signal when the detected level of carbon monoxide gas falls below the predetermined threshold level.

6. The carbon monoxide detection and expulsion system of claim 1, wherein the carbon monoxide detector is configured to measure the level of carbon monoxide gas in parts per million (PPM) and compare the measured level of carbon monoxide gas to a predetermined PPM threshold level.

7. The carbon monoxide detection and expulsion system of claim 6, wherein the carbon monoxide detector is configured to output a signal to the exhaust fan to be turned on or off based on whether the measured level of carbon monoxide gas exceeds the predetermined PPM threshold level.

8. The carbon monoxide detection and expulsion system of claim 1, wherein the exhaust fan is configured to have a cubic feet per minute (CFM) flow capacity that is determined based on a square footage of an area being protected.

9. The carbon monoxide detection and expulsion system of claim 1, wherein the exhaust fan is configured to be ceiling-mounted or wall-mounted.

10. The carbon monoxide detection and expulsion system of claim 1, wherein the exhaust fan is any or all of moisture-resistant, steam-resistant, and mold-resistant.

11. The carbon monoxide detection and expulsion system of claim 1, further comprising a sound alarm connected to the carbon monoxide detector, the carbon monoxide detector configured to send an activation signal to the sound alarm when the detected level of carbon monoxide gas exceeds the predetermined threshold level.

12. The carbon monoxide detection and expulsion system of claim 1, further comprising a power supply relay module connected to the carbon monoxide detector and the exhaust fan, wherein the carbon monoxide detector sends the activation signal to the power supply relay module, and wherein the power supply relay module is configured to send the activation signal to the exhaust fan.

13. A method of detecting and expelling carbon monoxide, the method comprising:
detecting a level of carbon monoxide;
comparing the detected level of carbon monoxide to a threshold level;
turning on an exhaust fan when the detected level of carbon monoxide exceeds the threshold level; and
expelling the carbon monoxide when the detected level of carbon monoxide exceeds the threshold level.

14. The method according to claim 13, wherein detecting the level of carbon monoxide includes detecting the level of carbon monoxide within an enclosed volume.

15. The method according to claim 14, wherein comparing the detected level of carbon monoxide to the threshold level includes comparing the detected level of carbon monoxide within the enclosed volume to the threshold level.

16. The method according to claim 13, wherein turning on the exhaust fan includes sending an activation signal to the exhaust fan when the detected level of carbon monoxide exceeds the threshold level.

17. The method according to claim 13, further comprising activating a sound alarm when the detected level of carbon monoxide exceeds the threshold level.

18. The method according to claim 13, wherein detecting the level of carbon monoxide includes measuring the level of carbon monoxide in parts per million.

19. The method according to claim 13, further comprising turning off the exhaust fan when the detected level of carbon monoxide falls below the threshold level.

20. The method according to claim 19, wherein turning off the exhaust fan includes sending a deactivation signal to the exhaust fan when the detected level of carbon monoxide falls below the threshold level.
